(19) United States
(12) Patent Application Publication
(10) Pub. No.: US 2020/0306502 A1
(43) Pub. Date: Oct. 1, 2020

(54) Bladder Catheter and Method for Manufacturing a Bladder Catheter

(71) Applicant: Saxonia R+D GmbH & Co. KG, Radeberg (DE)

(72) Inventors: Rudolph Lüning, Dresden (DE); Larry Purcell, Radeberg (DE); Manfred Wirth, Dresden (DE)

(21) Appl. No.: 16/830,281

(22) Filed: Mar. 26, 2020

(30) Foreign Application Priority Data
Mar. 26, 2019 (DE) 102019107707.9

(51) Int. Cl. A61M 25/00; A61M 25/10

(52) U.S. Cl. CPC A61M 25/0017; A61M 25/0045; A61M 25/1002; A61M 2025/1061; A61M 25/007; A61M 25/0009; A61M 2025/0019; A61M 25/0026

(57) ABSTRACT

Urinary catheters comprising a tube having an opening at the distal end, and at least one catheter eye that is to be positioned in the bladder, the tube comprising at least one wall region that can be inflated or filled relative to a wall region of the tube. The catheters can be used as permanent catheters that are retained securely in the bladder and completely empty said bladder. The urinary catheter consists of a plurality of layers that are applied in succession and are made of a rubber-like material. The wall region is at least one wall region of the tube, the wall region being at least two mutually separate layers made of the rubber-like material of the wall region. A component of the wall of the tube is at least one channel for inflating or filling or cleaning the wall region, so that the tube is integral.

### CROSS REFERENCE TO RELATED APPLICATIONS

This application claims the priority of DE 102019107707.9 filed on 2019 Mar. 26; this application is incorporated by reference herein in its entirety.

### BACKGROUND

The invention relates to urinary catheters that in each case comprise a tube having an opening at the distal end, and at least one catheter eye that is to be positioned in the bladder, the tube comprising at least one wall region that can be inflated or filled relative to a wall region of the tube, as well as to a method for the production thereof.

Urinary catheters are used in medicine to drain urine from the bladder through the urethra (transurethrally) or the abdominal wall. A distinction is made between disposable catheters and permanent catheters. The latter are designed to remain in the urethra and/or bladder for a longer period of time and are used for the continuous or temporary discharge of urine from the bladder. Catheters are not only used for procedures affecting the urinary tract and/or the bladder or the prostate, but are very often used postoperatively if it is foreseeable that the patient will not be able to get up for some time or that an exact fluid balance is required.

Permanent catheters are usually designed as two-way catheters, the catheter tube having a second channel in addition to a channel for draining the urine, via which a balloon located at the end of the catheter in the bladder can be filled with fluid. Said balloon functions as the retainer of the urinary catheter in the bladder. Saline solution or glycerine or glycerine solution is primarily used as the fluid for filling.

Three-way catheters are also used, in which the catheter tube has a third channel having a discharge opening at the end of the catheter, through which, for example, a rinsing solution or a drug can be introduced into the bladder. The flushing solution leaves the bladder, like the urine, through an opening, also known as a catheter eye, through the main channel.

DE 20 2012 006 814 U1 contains a urinary catheter having a tube, the tube having an insertion portion for insertion into a bladder via a urethra and the tube having a connection portion for connecting connection elements, and wherein fluid from the urethra can be drained at the connection portion via the insertion portion. A closable valve is arranged in the connection portion in such a way that fluid can be discharged from the connection portion when the valve is closed. When the valve is closed, a full urine bag can be changed without urine leaking from the connection portion of the urinary catheter.

A reusable catheter is known from DE 20 2007 007 713 U1, which differs from the known catheters in that the catheter body has at least one third channel for applying at least one drug parallel to the catheter body having at least one discharge opening. Through this discharge opening, after the catheter has been inserted into the bladder, targeted application of drugs in the region of the urethra can take place via the third channel of the catheter.

A transurethral indwelling catheter for the treatment of functional disorders of the bladder and/or the prostate is known from DE 20 2011 104 675 U1. The indwelling catheter consists of a catheter tube, a urinary balloon and a prostate balloon having the appropriate inlets and discharges. The urine draining opening is located above and, due to anatomical reasons, the prostate balloon is located below the bladder balloon. The walls of the urinary and prostate balloons are designed in such a way that fluids containing drugs and stored in the balloons can diffuse into the bladder or prostate in a targeted manner. When filled, the prostate balloon has a maximum outer diameter of between 20 and 30 charr. The indwelling catheter should have a maximum indwelling time of 48 hours in the bladder. The disadvantage of this catheter is that it is used exclusively for application in males. In addition, it can be assumed that the disclosed balloon strength is substantially too low to hold the catheter in the bladder.

WO 2017/174 715 A1 discloses an improved urinary catheter having a catheter tube with a first connection at the distal end and at least one proximal catheter eye, which can also be used as a permanent catheter. For this purpose, there is a storage container located in the bladder, which is connected via a channel to a second connection as the distal end of the channel. Furthermore, the channel is connected to the urethra via at least one opening in the channel wall. The urinary catheter can be inserted into the bladder via the urethra (transurethrally) or into the bladder as a suprapubic urinary catheter via the abdomen. Embodiments for the realisation of the balloon-shaped retainer elements, storage containers and channels are not the subject of this publication.

WO 96 26 748 A2 relates to balloon catheters having an inflatable balloon. After the shaping process, said balloon is folded distally until the distal end abuts the inwardly tapering neck of the catheter body. These are interconnected using an adhesive connection. The inflation lumen can be formed by inserting a removable wire into the mould along the predetermined path of the inflation lumen. The inflation lumen can thus be an integral component of the catheter wall.

DE 25 40 385 A1 discloses a catheter for removing urine from the bladder of a patient who has an inflatable balloon. This can be made by coating the outer surface of the shaft of the catheter with bentonite or another water-soluble, separating mass during the production of the catheter and immersing the coated shaft in a latex solution. The catheter has a filling channel for inflating the balloon. The implementation thereof is not discussed in detail.

A balloon catheter system is known from WO 2005 023 153 A2 which can have a plurality of balloons arranged one inside the other. These can be filled with various media. This allows for a controlled expansion to place stents of different lengths and diameters.

### SUMMARY

The invention relates to urinary catheters that in each case comprise a tube having an opening at the distal end, and at least one catheter eye that is to be positioned in the bladder, the tube comprising at least one wall region that can be inflated or filled relative to a wall region of the tube. These are characterised in particular by the fact that said catheters can be used as permanent catheters that are retained securely in the bladder and completely empty said bladder. For this purpose, the urinary catheter consists of a plurality of layers that are applied in succession and are made of a rubber-like material. The wall region is at least one wall region of the tube, the wall region being at least two mutually separate layers made of the rubber-like material of the wall region of the tube. A component of the wall of the tube is at least one channel for inflating and/or filling and/or cleaning the wall region, so that the tube is integral, and wherein the tube comprises the interconnected layers, the at least one wall region, the mutually separate layers and the channel.

### DETAILED DESCRIPTION

The object of the invention specified in claims 1 and 10 is to provide a urinary catheter as a permanent catheter with a secure hold in the bladder and is intended for entirely emptying said bladder.

This object is achieved with the features listed in claims 1 and 11.

The urinary catheters comprising in each case a tube having an opening at the distal end, and at least one catheter eye that is to be positioned in the bladder, the tube comprising at least one wall region that can be inflated or filled relative to a wall region of the tube are characterised in particular in that said catheters can be used as permanent catheters that are retained securely in the bladder and completely empty said bladder.

For this purpose, the urinary catheter consists of a plurality of layers that are applied in succession and are made of a rubber-like material. The wall region is at least one wall region of the tube, the wall region being at least two mutually separate layers made of the rubber-like material of the wall region of the tube. A component of the wall of the tube is at least one channel for inflating and/or filling and/or cleaning the wall region, so that the tube is integral, and wherein the tube comprises the interconnected layers, the at least one wall region, the mutually separate layers and the channel.

The urinary catheter can be inserted into the bladder via the urethra (transurethrally) or into the bladder as a suprapubic urinary catheter via the abdomen. In this case, the tube is a hollow and flexible pipe through which the urine can be drained out of the bladder. For this purpose, said tube has the opening at the distal end.

In the case of the urinary catheter, the opening of the tube and an opening of the channel are located at the distal end and thus the end of the tube facing away from the body. These openings are located outside the body when the urinary catheter is in place. The opening of the tube may be closed and/or provided with a bag for collecting the urine from the bladder. The opening of the channel can be closed, such that the inflated wall region ensures secure retention of the urinary catheter.

The length of the tube of the urinary catheter can be selected individually. If the urinary catheter is intended for use in a male, the tube is longer than in the case of a urinary catheter for a female.

The catheter eye is an opening in the wall of the tube, through which urine from the bladder can reach the first opening, via the tube, after the urinary catheter has been inserted into the bladder. The catheter eye may be round to oval in shape.

Advantageously, the urinary catheter consists of a plurality of layers that are applied in succession and are made of a rubber-like material, the inflatable wall region being at least one wall region of the tube which has layers which are mutually separated by a removable separating means. The urinary catheter thus has the same circumference over its length. In particular, no elevations are present, such that the urinary catheter is easy to position.

In order to produce a urinary catheter having a tube with an opening at the distal end and at least one catheter eye to be positioned in the bladder, the tube having at least one wall region which is inflatable or fillable relative to a wall region of the tube, for example, at least one layer of a polymerisable and/or vulcanisable material can be applied to a support member, a removable separating means can be applied to a wall region of the layer, at least one further layer of a polymerisable and/or vulcanisable material can be applied to the layer and the separating means. After each application, the layers polymerise and/or vulcanise to a rubber-like material. For this purpose, the separating means can be a film and/or a powder and/or a layer of a material. The layers can be applied by dip application, wherein in order to achieve the at least one inflatable or fillable wall region and a desired thickness of the tube, multiple dip application is carried out into the fluid rubber-like material formed by polymerisation and/or vulcanisation.

Furthermore, the urinary catheter is characterised in that the inflated or filled wall region is a balloon which at least in part incompletely surrounds the tube in the bladder. The region of the tube not covered by the balloon has the catheter eye. The inflated or filled wall region as a balloon or torus can be designed as an open ring having a C-shaped cross section. The inflated or filled wall region functions as a retainer for the tube. In the inflated or filled state, the balloon is thus present as a torus on the tube, which torus retains the catheter eye in the bladder. The balloon incompletely surrounds the tube. The catheter eye is arranged in the region of the tube that is not covered with the balloon. Advantageously, this may primarily be directly above the sphincter of the urethra, such that the bladder can be emptied completely. No urine sink, comprising crystallising salts, remains. This prevents crystals from migrating between the tube and the urethra where they may cause painful rubbing and/or act as a breeding ground for bacteria.

The layers of the tube may consist of latex for example.

A urinary catheter having a tube with an opening at the distal end and at least one catheter eye to be positioned in the bladder can be manufactured with the following steps:
applying at least one first layer of a polymerisable and/or vulcanisable material to a support member,
polymerising and/or vulcanising the layer to form a tube of a rubber-like material which is closed on one side,
applying a removable separating means to a region of the tube,
placing a removable thread or yarn or wire so that it ends on or on top of or within the separating means and outside the tube as a distal end,
applying at least one second layer of a polymerisable and/or vulcanisable material to the tube, the removable separating means and the thread or the yarn or the wire,
polymerising and/or vulcanising the second layer,
removing the thread or yarn or wire by pulling out, so that a channel is present having a first opening towards the inflatable or fillable wall region from the second layer above the separating means and a second opening at the distal end of the tube, so that the wall region can be inflated or filled via the channel,
removing the tube from the support member, and
inserting the catheter eye into the tube.

Advantageous embodiments of the invention are given below.

In one embodiment, the inflated or filled wall region is a torus in the urethra completely surrounding the tube, wherein the wall region has at least one aperture to the urethra. The inflated wall region can be a retainer. The wall region can be filled with a fluid medium, which can enter the inner wall of the urethra via the aperture, which can thus be kept moist. The fluid medium can be or contain at least one drug. The torus is thus a storage container.

At least one removable thread or removable wire or removable yarn is optionally positioned in each case between layers so that the thread or the wire or the yarn ends in the mutually separate layers of the wall region and outside the distal end and the channel is a channel formed by removing the thread or the wire or the yarn. For this purpose, the thread may be a textile, fibres or filaments, the yarn may be a textile, or the wire may be a metal wire and/or a plastics wire.

In one embodiment, inflatable or fillable wall regions are simultaneously wall regions of the tube, the wall regions in each case being three mutually separate layers made of the rubber-like material of the wall region of the tube. The tube has at least two channels for inflating or filling the respective wall region, so that when the wall regions are inflated or filled, a first balloon is present in a second balloon, each C-shaped in cross section, and the catheter eye is located in the region of the tube that is not covered by the balloons. Furthermore, the wall of the second balloon arranged in the bladder has at least one aperture to the bladder. The wall region of the second balloon has the at least one aperture. This aperture allows for discharge of a fluid medium, which thus enters the bladder into the bladder. The fluid medium can be or contain at least one drug. The second balloon is thus a storage container.

Optionally, inflatable or fillable wall regions are simultaneously wall regions of the tube, the wall regions being at least two mutually separate layers made of the rubber-like material of the wall region of the tube. The tube has two channels for inflating or filling a first wall region and a second wall region spaced apart in the longitudinal direction of the tube, so that when the first wall region is inflated or filled a balloon having a C-shaped cross section is present in the bladder, and when the second wall region is filled a torus is present in the urethra, wherein the catheter eye is located in the region of the tube which is not covered by the balloon. The wall region of the torus has at least one aperture to the urethra, which allows for discharge of a fluid medium, which thus enters the urethra. The fluid medium can be or contain at least one drug. The torus is thus a storage container.

Optionally, inflatable or fillable wall regions are simultaneously wall regions of the tube, first wall regions in each case being at least three mutually separate layers and second wall regions being at least two mutually separate layers of the rubber-like material of the wall region of the tube. The tube has a first channel for inflating or filling a first wall region to form a first balloon in the bladder. Furthermore, the tube has a second channel for filling the second wall region to form a torus in the bladder and for filling a first wall region spaced apart in the longitudinal direction of the tube to form a second balloon surrounding the first balloon in the bladder. The first balloon and the second balloon each have a C-shaped cross section and the catheter eye is located in the region of the tube that is not covered by the balloons. In addition, the wall of the torus arranged in the urethra has at least one aperture to the urethra and the wall of the second balloon arranged in the bladder has at least one aperture to the bladder. The wall region of the second balloon and the torus therefore have at least one aperture in each case. These allow for discharge of a fluid medium, which thus enters the bladder and urethra. The fluid medium can be or contain at least one drug. The second balloon and the torus are thus a plurality of storage containers.

In one embodiment, at least one removable thread element or removable wire element or removable yarn element is positioned in each case such that the aperture is an aperture formed by removing the thread element or the wire element or the yarn element.

The outer layer of the tube is optionally at least in part a hydrophobic layer and/or at least in part a layer impregnated with at least one material.

In one embodiment, at least one tubular stent is located below the inflated wall region, towards the distal end. The stent can be formed from wire in the shape of a screw or as a grid.

The layers are layers realised by dip application. In order to achieve a desired thickness and to form the inflatable wall region, a multiple dip application is carried out into the fluid and rubber-like material which is formed after the multiple dip application by means of polymerisation and/or vulcanisation.

Before removing the thread or the yarn or the wire or before removing the tube from the support member, a removable separating means can be applied to a region of the second layer. A further removable thread or yarn or wire is positioned so that it ends on or on top of or within the separating means and outside the second layer as the distal end of the tube. At least one third layer of a polymerisable and/or vulcanisable material is applied to the tube having the first layer and the second layer, the removable separating means and the further thread or the removable yarn or the removable wire. The third layer is polymerised and/or vulcanised. The thread or the yarn or the wire and/or the further thread or the further yarn or the further wire are or is removed by pulling out, so that channels are present having first openings towards the inflatable or fillable wall regions from the second and the third layer above the separating means and second openings are provided at the distal end of the tube, so that the wall regions can be inflated or filled via the channels. The tube is removed from the support member and the catheter eye is inserted into the tube.

Applying the first and second layers or the first, second and third layers can advantageously be carried out by dip application into the polymerisable and/or vulcanisable material.

After removing the at least one thread or yarn or wire, the separating means can be removed via the channel or channels.

An embodiment of the invention is shown schematically in each case in the drawings and will be described in greater detail in the following.

### BRIEF DESCRIPTION OF THE DRAWINGS

In the drawings:

FIG. 1 shows a urinary catheter having a wall region inflated as a balloon,

FIG. 2 shows a urinary catheter having a first wall region inflated as a balloon and a second wall region filled as a balloon,

FIG. 3 shows a urinary catheter having a wall region inflated as a balloon and a filled torus, and

FIG. 4 shows a urinary catheter having a wall region inflated as a balloon and wall regions filled as a balloon and a torus.

### DETAILED DESCRIPTION OF THE PREFERRED EMBODIMENTS

A urinary catheter substantially consists of a tube 1 comprising an opening 2 at the distal end, at least one catheter eye 4 that is to be positioned in the bladder 3, and a retainer.

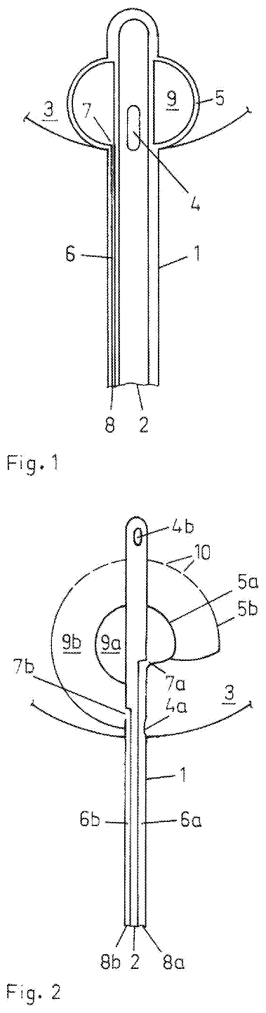

FIG. 1 is a schematic illustration of a urinary catheter having a wall region 5 inflated or filled as a balloon 9.

The end region or a region at the end of the tube 1 has an inflatable or fillable wall region 5, so that in the inflated or filled state a balloon 9 is present which incompletely surrounds the tube 1 as a retainer of the tube 1 in the bladder 3. The urinary catheter portion consists of a plurality of layers that are applied in succession and are made of a rubber-like material. The inflatable or fillable wall region 5 is at least one wall region 1 of the tube 1, the wall region 5 being at least two mutually separate layers of the rubber-like material of the wall region of the tube 1. A component of the wall of the tube 1 is furthermore at least one channel 6 for inflating and/or filling and/or cleaning the wall region 5, so that the tube 1 is integral. For inflating or filling the wall region 5, the tube 1 has the channel 6 having a first opening 7 towards the inflatable or fillable wall region 5 and a second opening 8 at the distal end of the tube 1 for inflating or filling the wall region 5. To form the channel 6, at least one removable thread or removable wire or removable yarn is optionally positioned between layers so that the thread or the wire or the yarn ends in the mutually separate layers of the wall region 5 and outside the distal end and the channel 6 is a channel 6 formed by removing the thread or the wire or the yarn. The channel 6 is thus a region of the wall of the tube 1. The inflated or filled wall region 5 is a balloon 9 as a retainer having a C-shaped cross section. The region of the tube 1 which is not covered by the balloon 9 has the catheter eye. Another catheter eye can be located above the wall region 5 in the bladder 3 and thus at the proximal end of the tube 1.

FIG. 2 is a schematic illustration of a urinary catheter having a first wall region 5 inflated as a balloon 9a and a second wall region 5 filled as a balloon 9b.

In one embodiment, inflatable or fillable wall regions 5a, 5b are simultaneously wall regions of the tube 1, the wall regions 5a, 5b each being in each case at least three mutually separate layers of the rubber-like material of the wall region of the tube 1 which are arranged parallel to one another. The tube 1 has at least two channels 6a, 6b for inflating or filling each wall region 5a, 5b, so that when the wall regions 5a, 5b are inflated or filled, a first balloon 9a is present in a second balloon 9b, each C-shaped in cross section, and the catheter eye 4 is located in the region of the tube 1 not covered by the balloons 9a, 9b. Thus, in the inflated or filled state, the balloons 9a, 9b which incompletely surround the tube 1 are present. In this case, both balloons 9a, 9b are used as retainers for the urinary catheter, in the form of a first balloon 9a and a second balloon 9b. Only when the second balloon 9b is empty does the first balloon 9a in particular act as a retainer of the urinary catheter. The first channel 6a is used for inflating the first balloon, in particular with air. The second balloon 9b is filled by means of the second channel 6b. For this purpose, the channels 6a, 6b comprise first openings 7a, 7b to the respective balloon 9a, 9b and second openings 8a, 8b at the distal end of the tube 1 as components of the wall of the tube 1 for supplying the respective medium. The second wall region 5b forming the second balloon 9b has apertures 10, so that in particular fluids in the second balloon 9b can enter the bladder. The second balloon 9b thus represents a storage container, in particular for a drug.

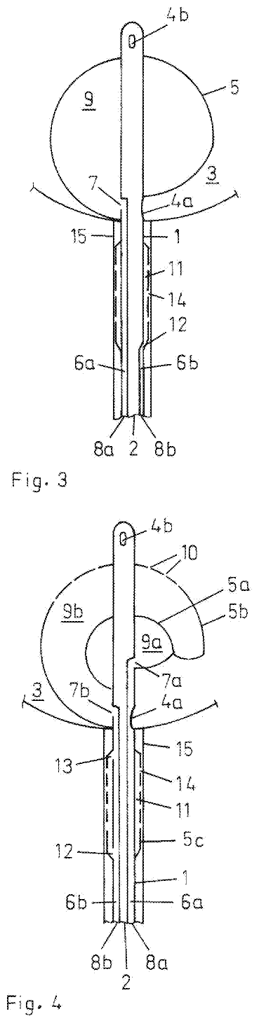

FIG. 3 is a schematic illustration of a urinary catheter having a wall region inflated as a balloon 9 and a filled torus 11.

A urinary catheter of a further embodiment has an inflatable and a fillable wall region 5, which are simultaneously wall regions of the tube 1. These are in each case at least two mutually separate layers of the rubber-like material of the wall region of the tube 1. The tube 1 has two channels 6a, 6b for inflating or filling a first wall region 5 and a second wall region spaced apart in the longitudinal direction of the tube 1, so that when the first wall region 5 is inflated or filled a balloon 9 having a C-shaped cross section is present in the bladder 3, and when the second wall region is filled a torus 11 is present in the urethra 15. The catheter eye 4 is located in the region of the tube 1 not covered by the balloon 9 and is thus a retainer of the urinary catheter. The wall region of the torus 11 has apertures 14 to the urethra 15, so that fluid and possibly at least one drug contained therein can enter the urethra 15.

FIG. 4 is a schematic illustration of a urinary catheter having a wall region 5a inflated as a balloon 9a and a wall regions 5b, 5c filled as a balloon 9b and a torus 11.

In the case of a urinary catheter of a further embodiment, the tube 1 can have a third inflatable wall region 5c having a third opening 12 and a fourth opening 13 to the second channel 6b in connection with the second balloon 9b, so that in the filled state a torus 11 surrounding the tube 1 at least in part is arranged in the urethra 15. The third wall region 5c has apertures 14. With the addition of fluid via the second channel 6b, fluid enters both the torus 11 and the second balloon 9b. Fluid and thus in particular at least one drug can therefore enter the bladder 3 via the apertures 10 of the second wall region 5b of the second balloon 9b. Simultaneously, fluid and thus also the drug can enter the urethra 15 via the apertures 14.

In further advantageous configurations of the embodiments, the tube 1 can be open at the proximal end or, in a further configuration with the tube 1 closed at the proximal end, a further catheter eye can be arranged as a second catheter eye 4b, wherein the catheter eye 4 located in the region of the tube 1 not covered by the balloons 9a, 9b is a first catheter eye 4a.

The tube 1 of the exemplary embodiment, the embodiments and the configurations consists of layers which are applied by dip application. To achieve a desired thickness and to form the at least one inflatable wall region 5, multiple dip application into the fluid rubber-like material formed after the multiple dip application by polymerisation and/or vulcanisation is carried out, from which material the tube 1 and thus the respective urinary catheter is then made.

### LIST OF REFERENCE NUMERALS

1 tube
2 opening of the tube
3 bladder
4 catheter eye
5 wall region
6 channel
7 first opening in the first and second channels
8 second opening in the first and second channels
9 balloon
10 aperture in the second wall region
11 torus
12 third opening in the second channel
13 fourth opening in the second channel
14 aperture in the third wall region
15 urethra

1. Urinary catheter having a tube (1) with an opening (2) at the distal end and at least one catheter eye (4) to be positioned in the bladder (3), the tube (1) having at least one wall region (5) which can be inflated or filled relative to a wall region of the tube (1), characterised in that the urinary catheter consists of a plurality of layers that are applied in succession and are made of a rubber-like material, in that the wall region (5) is at least one wall region (5) of the tube (1), the wall region (5) being at least two mutually separate layers of the rubber-like material of the wall region of the tube (1), in that a component of the wall of the tube (1) is at least one channel (6) for inflating and/or filling and/or cleaning the wall region (5), that the tube (1) is integral, and has the interconnected layers, the at least one wall region having the mutually separate layers and the channel (6), in that the inflated or filled wall region (5) is a balloon (9) in the bladder (3), which at least in part incompletely surrounds the tube (1), and in that the region of the tube (1) which is not covered by the balloon (9) has the catheter eye (4).

2. Urinary catheter according to claim 1, characterised in that the inflated or filled wall region (5) is a torus (11) in the urethra (15) completely surrounding the tube and in that the wall region (5) has at least one aperture (14) to the urethra (15).

3. Urinary catheter according to claim 1, characterised in that in each case at least one removable thread or removable wire or removable yarn is optionally positioned between layers so that the thread or the wire or the yarn ends in the mutually separate layers of the wall region (5) and outside the distal end, and the channel (6) is a channel (6) formed by removing the thread or the wire or the yarn.

4. Urinary catheter according to claim 1, characterised in that inflatable or fillable wall regions (5) are simultaneously wall regions (5) of the tube (1), the wall regions in each case being at least three mutually separate layers of the rubber-like material of the wall region of the tube (1), in that the tube (1) has at least two channels (6a, 6b) for inflating or filling the respective wall region (5a, 5b), so that when the wall regions (5a, 5b) are inflated or filled, a first balloon (9a) is present in a second balloon (9b), each being C-shaped in cross section, and the catheter eye (4) is located in the region of the tube (1) not covered by the balloons (9a, 9b), and in that the wall of the second balloon (9b) arranged in the bladder (3) has at least one aperture (10) to the bladder (3).

5. Urinary catheter according to claim 1, characterised in that inflatable or fillable wall regions (5) are simultaneously wall regions of the tube (1), the wall regions in each case being at least two mutually separate layers of the rubber-like material of the wall region (5) of the tube (1), in that tube (1) has two channels (6a, 6b) for inflating or filling a first wall region (5) and a second wall region spaced apart in the longitudinal direction of the tube (1), so that when the first wall region (5) is inflated or filled a balloon (9) having a C-shaped cross section is present in the bladder (3), and when the second wall region is filled a torus (11) is present in the urethra (15), wherein the catheter eye (4) is located in the region of the tube (1) that is not covered by the balloon (9), and in that the wall region of the torus (11) has at least one aperture (14) to the urethra (15).

6. Urinary catheter according to claim 1, characterised in that inflatable or fillable wall regions (5) are simultaneously wall regions of the tube (1), first wall regions (5) in each case having at least three mutually separate layers and second wall regions having at least two mutually separate layers in each case made of the rubber-like material of the wall region of the tube (1), in that the tube (1) has a first channel (6a) for inflating or filling a first wall region (5) to form a first balloon (9a) in the bladder (3), in that the tube (1) has a second channel (6b) for filling the second wall region to form a torus (11) in the bladder (15) and for filling a first wall region (5) spaced apart in the longitudinal direction of the tube (1) to form a second balloon (9b) surrounding the first balloon (9a) in the bladder (3), in that the first balloon (9a) and the second balloon (9b) are each C-shaped in cross section and the catheter eye (4) is located in the region of the tube (1) not covered by the balloons (9a, 9b), and in that the wall of the torus (11) arranged in the urethra (15) has at least one aperture (14) to the urethra (15) and the wall of the second balloon (9b) arranged in the bladder 3 has at least one aperture (10) to the bladder (3).

7. Urinary catheter according to claim 2, characterised in that in each case at least one removable thread element or a removable wire element or a removable yarn element is positioned such that the aperture (10, 14) is an aperture (10, 14) formed by removing the thread piece or the wire piece or the thread piece.

8. Urinary catheter according to claim 1, characterised in that the outer layer of the tube (1) is at least in part a hydrophobic layer and/or in that the outer layer of the tube (1) is at least in part a layer impregnated with at least one material.

9. Urinary catheter according to claim 1, characterised in that at least one tubular stent is located below the inflated wall region (5) towards the distal end.

10. Urinary catheter according to claim 1, characterised in that the layers are layers realised by means of repeated dip application into a reservoir of the rubber-like material which is thereby formed.

11. Method for producing a urinary catheter having a tube (1) with an opening (2) at the distal end and at least one catheter eye (4) to be positioned in the bladder (3) with the following steps:
applying at least one first layer of a polymerisable and/or vulcanisable material to a support member,
polymerising and/or vulcanising the layer to form a tube (1) of a rubber-like material which is closed on one side,
applying a removable separating means to a region of the tube (1),
placing a removable thread or yarn or wire so that it ends on or on top of or within the separating means and outside the tube (1) as a distal end,
applying at least one second layer of a polymerisable and/or vulcanisable material to the tube (1), the removable separating means and the thread or the yarn or the wire,
polymerising and/or vulcanising the second layer,
removing the thread or yarn or wire by pulling out, so that a channel (6) is present having a first opening (7) towards the inflatable or fillable wall region (5) from the second layer above the separating means and a second opening (8) at the distal end of the tube (1), so that the wall region (5) can be inflated or filled via the channel (6),
removing the tube (1) from the support member, and
inserting the catheter eye (4) into the tube (1).

12. Method according to claim 11, characterised
in that before removing the thread or the yarn or the wire or before removing the tube (1) from the support member, a removable separating means is applied to a region of the second layer,
in that a further removable thread or yarn or wire is positioned such that it ends on or on top of or within the separating means and outside the second layer as the distal end of the tube (1),
in that at least one third layer of a polymerisable and/or vulcanisable material is applied to the tube (1) having the first layer and the second layer, the removable separating means and the further thread or the removable yarn or the removable wire,
in that the third layer is polymerised and/or vulcanised,
in that the thread or the yarn or the wire and/or the further thread or the further yarn or the further wire are or is removed by pulling out, so that channels (6a, 6b) are present having first openings (7a, 7b) towards the inflatable or fillable wall regions (5a, 5b) from the second and the third layer above the separating means, and second openings (8a, 8b) are present at the distal end of the tube (1), so that the wall regions (5a, 5b) can be inflated or filled via the channels (6a, 6b),
in that the tube (1) is removed from the support member, and
in that the catheter eye (4) is inserted into the tube (1).

13. Method according to claim 11, characterised in that applying the first and second layers or the first, second and third layers can be carried out by dip application into the polymerisable and/or vulcanisable material.

14. Method according to claim 11, characterised in that after removing the at least one thread or yarn or wire, the separating means is removed via the channel (6) or the channels.
